(19) United States
(12) Patent Application Publication
(10) Pub. No.: US 2019/0247052 A1
(43) Pub. Date: Aug. 15, 2019

(54) Assembly and Device for Left Atrial Appendage Occlusion

(71) Applicant: Arvin Narula, San Diego, CA (US)

(72) Inventor: Arvin Narula, San Diego, CA (US)

(21) Appl. No.: 16/274,958

(22) Filed: Feb. 13, 2019

Related U.S. Application Data
(60) Provisional application No. 62/629,940, filed on Feb. 13, 2018.

(51) Int. Cl. A61B 17/12; A61B 17/00

(52) U.S. Cl. CPC A61B 17/12122; A61B 17/00234; A61B 17/1219; A61B 17/0057; A61B 2017/00632; A61B 2017/00243; A61B 2017/00619; A61B 2017/1205; A61B 17/1214

(57) ABSTRACT

A left atrial appendage occlusion medical device includes a carrier and a hydrogel polymer. The carrier is for placement within a left atrial appendage of a heart. The carrier can be a spherical shaped structure of a shape memory material or a framing coil of metal material, among other possible examples. The hydrogel polymer is carried by the carrier and hence is placed within the left atrial appendage along with the carrier. The hydrogel polymer is capable of expanding in size when the hydrogel polymer comes into contact with blood fluid. In use, upon placement of the left atrial appendage occlusion medical device within the left atrial appendage, the hydrogel polymer expands in size when it comes into contact with blood fluid.

### CROSS-REFERENCE TO RELATED APPLICATIONS

This application claims the benefit of U.S. provisional patent application No. 62/629,940 filed Feb. 13, 2018, the entire contents of which are hereby incorporated by reference.

### TECHNICAL FIELD

This disclosure relates generally to techniques for closing the left atrial appendage of the heart, and relates particularly to medical devices and methods employed to close the left atrial appendage of the heart.

### BACKGROUND

The left atrial appendage is a reservoir for blood spanning from the left atrium. When in a normal heart rhythm, the left atrial appendage can function as a pop off valve for extra blood flow. But when in atrial fibrillation the left atrial appendage can lead to clot formation and even stroke in some cases. One estimate is that 90% of clots arise from the left atrial appendage.

Atrial fibrillation is an irregular heart rhythm where the top chambers of the heart are not fully contracting. This can lead to blood pooling particularly in the left atrial appendage, which leads to formation of clot. This clot can lead to embolization and lead to stroke, heart attack, or major organ damage and even death in some cases.

The standard medical therapy often involves blood thinners. This can limit the amount of clot formation in the left atrial appendage. But the side effects of blood thinners are bleeding, and the most feared complication is intracranial bleeds. One downside of blood thinners is poor compliance particularly in patients with atrial fibrillation. Since not all patients are candidates for long term blood thinners, medical devices have been developed to occlude the left atrial appendage.

Currently, one FDA-approved product known as the Watchman device is available for left atrial appendage occlusion. U.S. Pat. No. 6,689,150 is related to this device. One shortcoming with this device is the left atrium is a very thin structure and the Watchman device has barbs that can potentially perforate the heart muscle.

There are other devices that have been attempted to close the left atrial appendage, but given the location of the pouch, all the devices can come with significant risks. U.S. Pat. No. 6,488,689 involves lassoing the left atrial appendage closed, but this can have risks of device failure, atrial and ventricular puncture.

### SUMMARY

In an embodiment, a left atrial appendage occlusion medical device may include a carrier and a hydrogel polymer. The carrier is for placement within a left atrial appendage of a heart. The hydrogel polymer is carried by the carrier and hence is placed within the left atrial appendage along with the carrier. The hydrogel polymer is capable of expanding in size when the hydrogel polymer comes into contact with blood fluid. In use, upon placement of the left atrial appendage occlusion medical device within the left atrial appendage, the hydrogel polymer expands in size when it comes into contact with blood fluid.

In another embodiment, a method of placing the left atrial appendage occlusion medical device within the left atrial appendage of the heart may include the use of a delivery sheath, a dilator, a transseptal needle, and the left atrial appendage occlusion medical device.

### BRIEF DESCRIPTION OF DRAWINGS

The following detailed description of an embodiment is set forth with reference to the accompanying drawings, in which:

FIG. 1 is a schematic depicting access of a delivery sheath in the femoral vein, through the fossa ovalis into the left atrium, and depicting a left atrial appendage occlusion device according to an embodiment sitting fixed in the left atrial appendage;

FIG. 2 is a schematic depiction of the left atrial appendage occlusion device of FIG. 1 sitting fixed in the left atrial appendage with an expanded hydrogel polymer growing to the size of the structure;

FIG. 3 is a schematic depiction of an inner sphere of the left atrial appendage occlusion device that is a fixed size surrounded the hydrogel polymer of the left atrial appendage occlusion device, which can then grow when in contact with fluid;

FIG. 4 is a schematic depiction of a dilator and sheath that can be employed to deliver the left atrial appendage occlusion device;

FIG. 5 is a schematic depiction of a transseptal needle to cross the interatrial septum;

FIG. 6 is a schematic depiction of another embodiment of the left atrial appendage occlusion device; and

FIG. 7 is a schematic depiction of yet another embodiment of the left atrial appendage occlusion device.

### DETAILED DESCRIPTION OF EMBODIMENTS

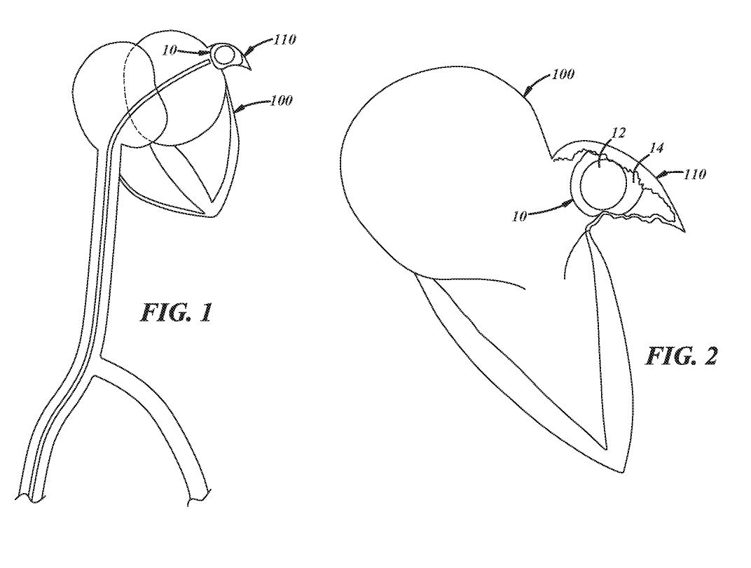

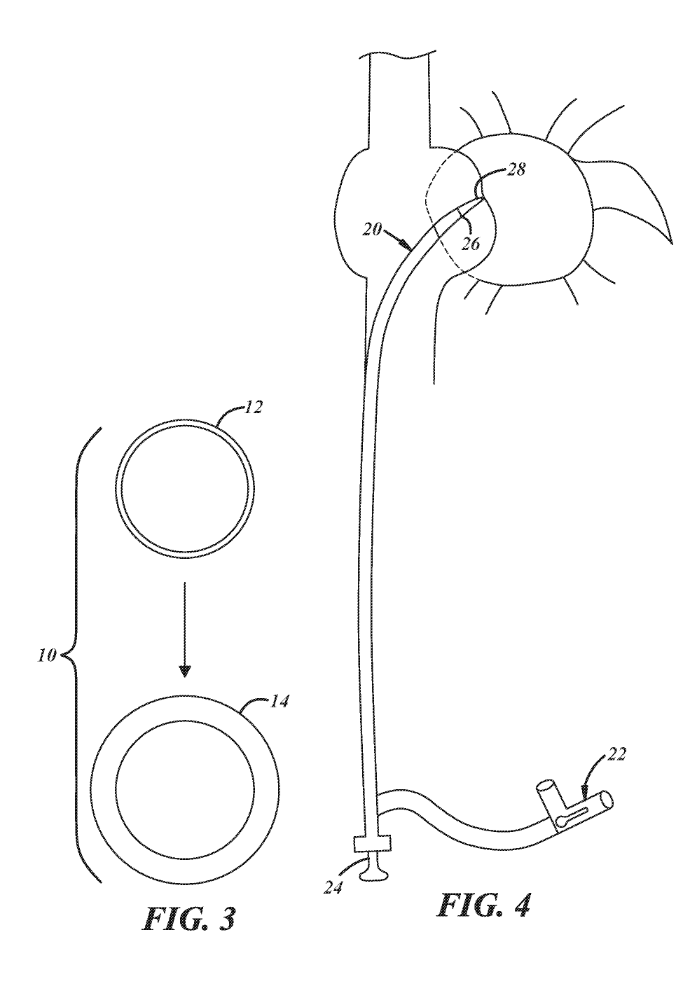

The present disclosure serves to overcome some of the shortcomings and risks involved with current technologies of left atrial appendage occlusion. To date, there is no known medical device involving left atrial appendage occlusion that involves growth of a soft hydrogel polymer to promote endothelialization of the device and adequate compression to avoid embolization. In the embodiment presented by FIGS. 1-3, a left atrial appendage occlusion medical device 10 includes a carrier 12 in the form of an expandable spherically-shaped structure and includes a hydrogel polymer 14. The spherically-shaped structure can grow from a first size to a second size when prompted, and can be composed of a shape memory material such as a shape memory alloy. The first size can be suitable when the spherically-shaped structure is in the midst of delivery via a delivery sheath 20, and the second size can be suitable when the spherically-shaped structure is situated in place within the left atrial appendage 110. The spherically-shaped structure may be available in different dimensions for differing sizes of a particular patient's left atrial appendage 110. The hydrogel polymer 14 is carried by the carrier 12 and expands and grows in size to fit the specific sizes of a left atrial appendage 110 of a heart 100. The hydrogel polymer 14 can be in gel form and can be carried by the carrier 12 as a coating. Expansion and growth in size of the hydrogel polymer 14 can be prompted by contact with blood fluid in the left atrial appendage 110. The hydrogel polymer 14 then promotes endothelial growth over the carrier 12, thus occluding the left atrial appendage 110.

With reference to FIG. 4, the delivery sheath 20 carries and is employed to deliver the left atrial appendage occlusion medical device 10 and prevents the hydrogel polymer 14 from expanding prematurely. The delivery sheath 20 is advanced from the femoral vein into the left atrium with a transseptal puncture. The delivery sheath 20 can be double braided and hydrophilic. The double braided sheath prevents kinking with excessive torqueing. The delivery sheath 20 can have a fixed angle between approximately 50-80 degrees in order to have back wall support in the right atrium; other angles are possible. This will assist in crossing the septum as well as aiming the delivery sheath 20 into the left atrial appendage 110. The delivery sheath 20 also has a side port and a three-way stop cock 22 for flushing. An inner lumen of the delivery sheath 20 initially serves to have enough room for a dilator 24 to cross the interatrial septum. The dilator 24 can have an echogenic marker 26. The echogenic marker 26 can be positioned approximately 10 mm from a leading tip in order to prevent the dilator 24 from being advanced too far. The dilator tip can have enough room for an 0.032″ wire and a transseptal needle 28.

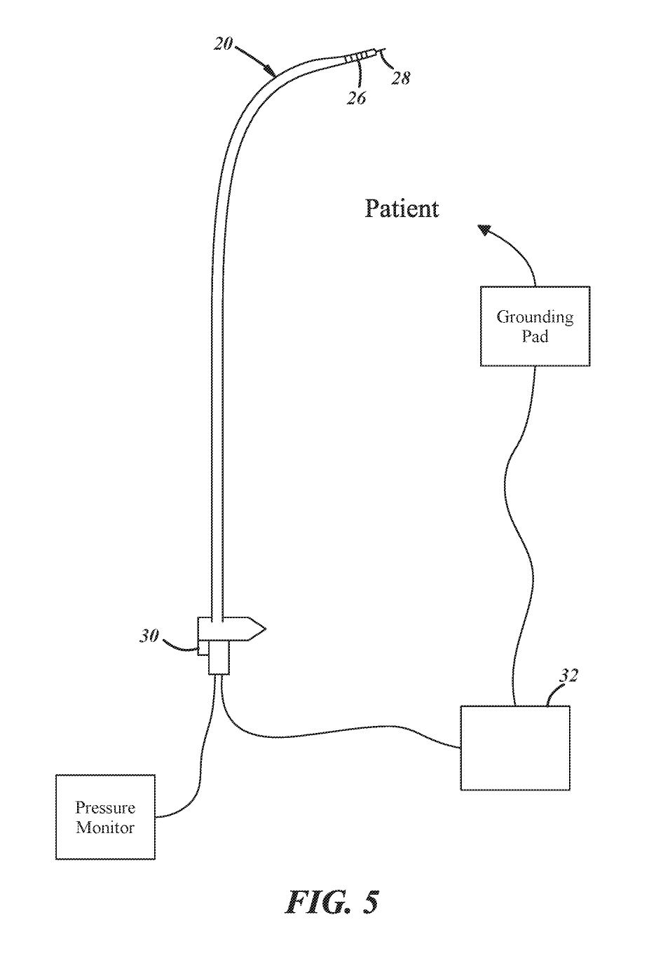

With reference now to FIGS. 4 and 5, the transseptal needle 28 fits inside the lumen of the dilator 24. The shape consists of a 50-80 degree angle, but could have other degree values. The tip of the transseptal needle 28 reaches outside of the dilator 24 and performs the puncture. The tip of the transseptal needle 28 has radiofrequency to bovie through material such as the interatrial septum. There can be indicator markers between approximately 2-10 mm that are echogenic in order to visualize the location of the needle tip under ultrasound. The bovie gets activated by a button 30 that is on the handle of the transseptal needle 28 in order to burn in a controlled fashion. The bovie can be hooked up to a radiofrequency generator 32 that is also hooked up to a grounding pad on the patient.

Once the delivery sheath 20 is inside the left atrial appendage 110, the spherically-shaped structure of the shape memory material is inserted inside the delivery sheath 20 and delivered into the desired location. The hydrogel polymer 14 expands when it comes into contact with blood fluid. This serves two purposes: i) it anchors the left atrial appendage occlusion medical device 10 well into the left atrial appendage 110 to prevent embolization and ii) the hydrogel polymer 14 exposed to the left atrium will promote endothelialization of the left atrial appendage occlusion medical device 10 and eventually closure of the left atrial appendage 110 from the left atrium.

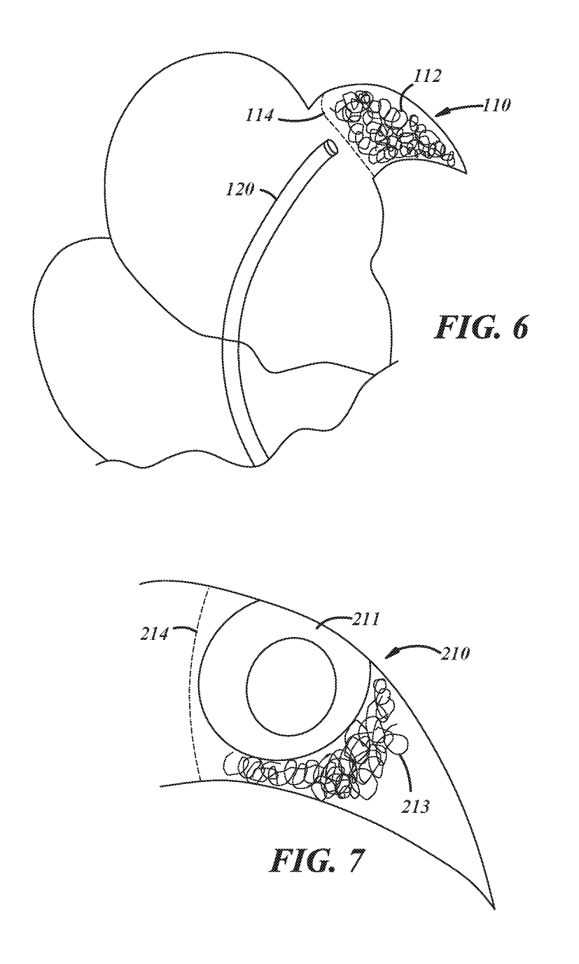

Another embodiment of the left atrial appendage occlusion medical device is presented in FIG. 6 and denoted by the numeral 110 in that figure. The left atrial appendage occlusion medical device 110 includes the carrier and the hydrogel polymer 114, as previously presented. But in this embodiment the carrier is in the form of a framing coil 112. The framing coil 112 can carry the hydrogel polymer 114 at its exterior. The framing coil 112 can be composed of a metal material such as a platinum material or the like, and can take a wire-like formation. As before, this embodiment of the left atrial appendage occlusion medical device 110 is delivered by the delivery sheath 120.

Yet another embodiment of the left atrial appendage occlusion medical device is presented by FIG. 7 and denoted by the numeral 210 in that figure. The left atrial appendage occlusion medical device 210 includes the carrier and the hydrogel polymer 214, as previously presented. But in this embodiment the carrier includes a first carrier 211 and a second carrier 213. The first carrier 211 is in the form of the expandable spherically-shaped structure of a shape memory material, as previously described. The second carrier 213 is in the form of the framing coil of metal material, as previously described. Both the first and second carriers 211, 213 carry the hydrogel polymer 214.

It is to be understood that the foregoing description is of one or more preferred exemplary embodiments of the invention. The invention is not limited to the particular embodiment(s) disclosed herein, but rather is defined solely by the claims below. Furthermore, the statements contained in the foregoing description relate to particular embodiments and are not to be construed as limitations on the scope of the invention or on the definition of terms used in the claims, except where a term or phrase is expressly defined above. Various other embodiments and various changes and modifications to the disclosed embodiment(s) will become apparent to those skilled in the art. All such other embodiments, changes, and modifications are intended to come within the scope of the appended claims.

As used in this specification and claims, the terms “for example,” “for instance,” and “such as,” and the verbs “comprising,” “having,” “including,” and their other verb forms, when used in conjunction with a listing of one or more components or other items, are each to be construed as open-ended, meaning that the listing is not to be considered as excluding other, additional components or items. Other terms are to be construed using their broadest reasonable meaning unless they are used in a context that requires a different interpretation.

1. A left atrial appendage occlusion medical device, the medical device comprising:
a carrier for placement within a left atrial appendage of a heart; and
a hydrogel polymer carried by said carrier and placed within the left atrial appendage with said carrier, said hydrogel polymer being expandable in size upon contact with blood;
wherein, upon placement of the left atrial appendage occlusion medical device within the left atrial appendage, said hydrogel polymer expanding in size upon contact with blood.

2. The left atrial appendage occlusion medical device as set forth in claim 1, wherein said carrier is composed of a shape memory material.

3. The left atrial appendage occlusion medical device as set forth in claim 2, wherein said carrier is a generally spherical shaped structure.

4. The left atrial appendage occlusion medical device as set forth in claim 1, wherein said carrier is a framing coil composed of a metal material.

5. The left atrial appendage occlusion medical device as set forth in claim 1, wherein said carrier comprises a first carrier and a second carrier, said first carrier is a generally spherical shaped structure composed of a shape memory material, said second carrier is a framing coil composed of a metal material, said first carrier carrying said hydrogel polymer, and said second carrier carrying said hydrogel polymer.

6. A method of placing the left atrial appendage occlusion medical device within the left atrial appendage of the heart, the method comprising the use of a delivery sheath, a dilator, a transseptal needle, and the left atrial appendage occlusion medical device as set forth in claim 1.
